(19) United States
(12) Patent Application Publication
(10) Pub. No.: US 2021/0244302 A1
(43) Pub. Date: Aug. 12, 2021

(54) METHODS TO ESTIMATE THE BLOOD PRESSURE AND THE ARTERIAL STIFFNESS BASED ON PHOTOPLETHYSMOGRAPHIC (PPG) SIGNALS

(71) Applicant: Evonik Operations GmbH, Essen (DE)

(72) Inventors: Rosario LIZIO, Dieburg (DE); Sara LIEBANA VIÑAS, Aschaffenburg (DE); Abdelhak ZOUBIR, Darmstadt (DE); Michael MUMA, Darmstadt (DE); Tim SCHAECK, Sindelfingen (DE)

(73) Assignee: Evonik Operations GmbH, Essen (DE)

(21) Appl. No.: 17/045,715

(22) PCT Filed: Apr. 18, 2019
(86) PCT No.: PCT/EP2019/060121

§ 371 Date: Oct. 6, 2020

(30) Foreign Application Priority Data
Apr. 23, 2018 (EP) 18168758.3

(51) Int. Cl. A61B 5/024; A61B 5/0285; A61B 5/00; A61B 5/107; A61B 5/02; A61B 5/021; G16H 50/30

(52) U.S. Cl. CPC A61B 5/02416; A61B 5/0285; A61B 5/02405; A61B 5/7203; A61B 5/742; A61B 5/681; A61B 5/1072; A61B 5/02007; A61B 5/02116; A61B 5/7405; G16H 50/30; A61B 5/7239

(57) ABSTRACT

The present invention relates to a method to estimate the blood pressure and the arterial stiffness based on photoplethysmographic (PPG) signals. New algorithms have been developed and validated based on PPG signals to analyze the cardiovascular condition of a person by estimating cardiovascular parameters. With the present invention a method for measuring one or more cardiovascular parameters in a subject based on PPG signals is provided.

The present invention relates to a method to estimate the blood pressure and the arterial stiffness based on photoplethysmographic (PPG) signals. New algorithms have been developed and validated based on PPG signals to analyze the cardiovascular condition of a person by estimating cardiovascular parameters. With the present invention a method for measuring one or more cardiovascular parameters in a subject based on PPG signals is provided.

Photoplethysmographic (PPG) sensors can be found in a number of different devices. Not only are they built into consumer goods such as wrist-type fitness trackers but also into devices used by medical professionals. The sensors are mostly used to either estimate the pulse rate or the oxygen saturation in the blood.

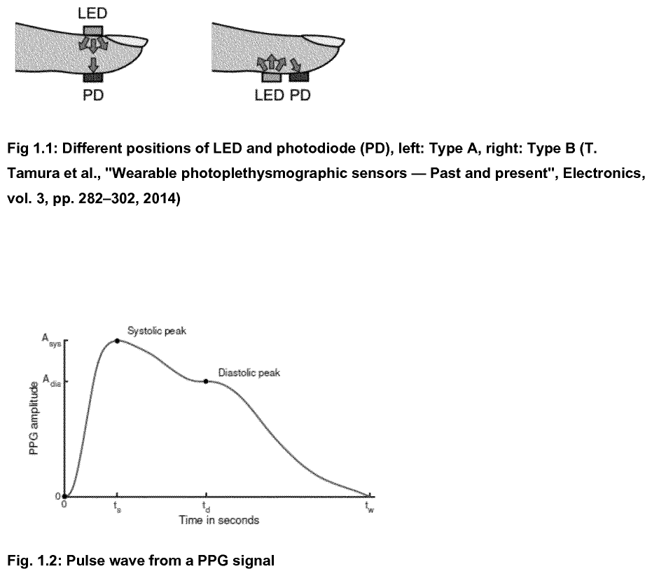

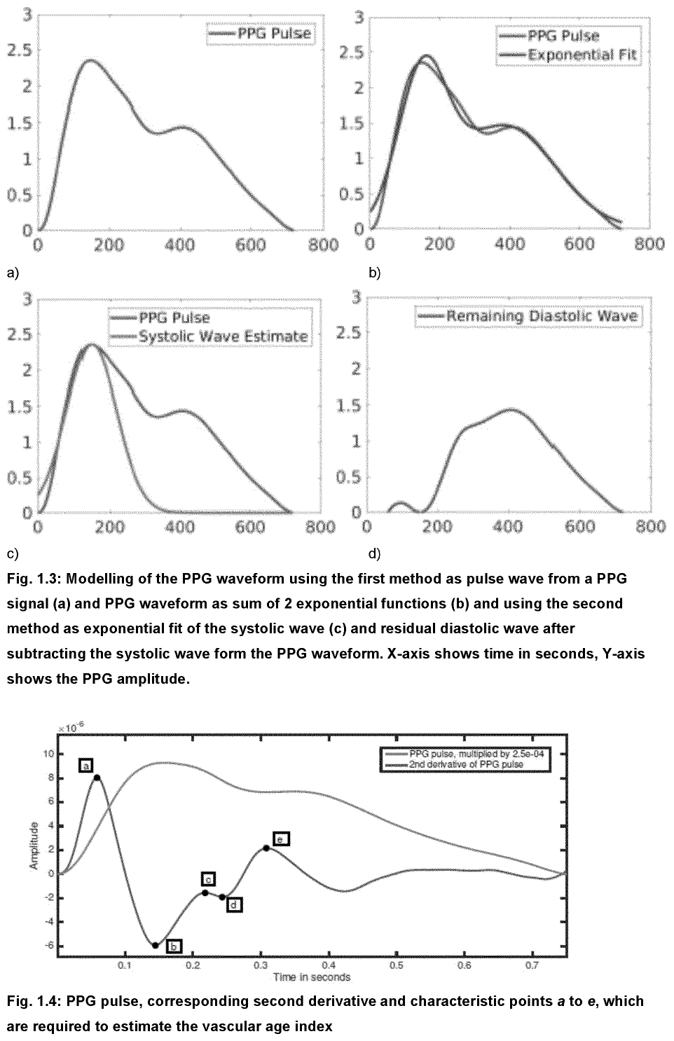

A plethysmograph is an instrument that measures changes in volume of an organ and is basically an optical sensor. The term photoplethysmography usually refers to the measurement of volume changes in arteries and arterioles due to blood flow. There are different kinds of PPG sensors. Some are placed at the fingertip, some at the wrist and other sites such as the ear lobe are also possible. The sensor itself consists of a light emitting diode (LED) that emits light onto the skin and of a photodiode. This diode is usually placed next to the LED, detecting light that is reflected (Type B). For finger sensors, the photodiode can also be placed at the opposite end of the finger, measuring the light that travels through the finger (Type A). FIG. 1.1 shows the different types.

### Relation Between Cardiovascular Parameters and Arterial Stiffness

With increasing age, the blood vessels usually become stiffer compared to those of a young person. This phenomenon occurs primarily because elastin in blood vessels' walls deteriorates and is replaced by collagen, which is less flexible. The increased stiffness causes the blood to travel faster through the vessels, therefore arterial stiffness is strongly correlated to the pulse wave velocity PWV. If a person's arterial stiffness is higher than the normal value for their age, this is a determinant of hypertension, i.e. increased systolic and diastolic blood pressure. As mentioned above, hypertension is an increasingly large problem, thus arterial stiffness is of interest as well.

Since increased arterial stiffness can be detected before hypertension occurs, this allows to start treatment or behavioral changes early, possibly avoiding hypertension. It is also well known that atherosclerotic plaques and aneurysms involve changes in vessel wall properties and therefore their stiffness (M. McGarry et al., “In vivo repeatability of the pulse wave inverse problem in human carotid arteries”, J. of biomechanics, vol. 64, pp. 136-144, 2017). Also in this case, an accurate arterial stiffness measurement, in particular its variation, would improve diagnosis and monitoring of the connected diseases. Various cardiovascular parameters can be analyzed to gain information about a person's cardiovascular health.

Blood pressure (BP) denotes the pressure that the blood traveling through a large artery exerts onto its walls. Hypertension is a major risk factor for multiple diseases, such as stroke and end-stage renal disease, and overall mortality. By the year 2025, it is expected that the number of people across the world who are hypertensive will have risen to 1.56 billion. If the condition is detected early and treated properly, the risk of disease can be decreased significantly. Therefore, it is important to measure BP regularly in order to detect abnormal changes. Besides this, a change of lifestyle can often decrease BP and prevent hypertension, provided that a tendency towards it is detected early. Currently, there exist several different approaches to measure BP. The most common device is an inflatable cuff that is placed at the patient's arm and that applies pressure onto the brachial artery. While this allows an accurate measurement, it is perceived as inconvenient by some patients and it requires a visit to a doctor or the purchase of a device. Other approaches are invasive, such as intravenous cannula that are placed inside an artery. Those are only used in a clinical context, e.g. during a surgery. A PPG signal can be obtained comfortably, continuously and at low cost. Extracting information about BP can serve an important purpose: As it is easy to obtain at home, this could warn a person early and advise them to seek medical advice.

Pulse wave velocity (PWV) describes the velocity of blood that travels through a person's arteries and is used as a measure of arterial stiffness. The most precise devices to measure PWV perform a carotid-femoral measurement. For this measurement, one tonometer is placed at the carotid artery which is located at the neck and a second tonometer is placed at the femoral artery at the upper leg. Those tonometers measure the pressure pulse waves of the arteries. From the time difference between the signals and the distance between the tonometers, PWV can be calculated. A more convenient way to estimate the PWV is by using two PPG sensors at a known distance or one PPG sensor and an electrocardiogram (ECG) and to calculate PWV from the time difference between the signals. PWV can also be measured with only one blood pressure cuff. This technique is used by the “Mobil-OGraph PWA” which is a clinical device by I.E.M. GmbH that has been used as a reference device in the experimental setup.

Vascular age index (AgIx) is a cardiovascular parameter that gives information on the age condition of the arteries. It can be determined with devices that uses an inflatable cuff. In the literature the AgIx as given from the second derivative of the PPG pulse wave form.

Augmentation index (AIx) is a cardiovascular parameter that is usually obtained from a pressure pulse wave and can be measured at a large artery with a device that uses an inflatable cuff. In contrast, the PPG sensor is unable to measure pressure and only detects volume changes in very small arteries and arterioles. Just like arterial stiffness, the augmentation index increases with age and can be used to estimate the risk of suffering from a cardiovascular disease in the future.

Heart rate variability (HRV) describes the variation in the time interval between heartbeats and is usually calculated from an ECG. Normally, the HRV is determined from the PPG signal based on determining the locations of the systolic feet.

Different systems for measuring blood pressure as an alternative to inflatable cuffs have been described, such as in WO 2015/066445 A1 where a system and method for measuring and monitoring blood pressure is provided. The system includes a wearable device and a tonometry device coupled to the wearable device. The Tonometry device is configured to compress a superficial temporal artery (STA) of a user. A sensor pad is attached to the wearable device adjacent the tonometry device. A blood pressure sensor is integrated within the sensor pad for continuous, unobtrusive blood pressure monitoring.

WO 2015/193917 A2 discloses a method and system for cuff-less blood pressure (BP) measurement of a subject. The method includes measuring, by one or more sensors, a local pulse wave velocity (PWV) and/or blood pulse waveforms of an arterial wall of the subject. Further, the method includes measuring, by an ultrasound transducer, a change in arterial dimensions over a cardiac cycle of the arterial wall of the subject. The arterial dimensions include an arterial distension and an end-diastolic diameter. Furthermore, the method includes measuring, by a controller unit, BP of the subject based on the local PWV and the change in arterial dimensions.

Further, different approaches for measuring one or more cardiovascular parameters have been proposed. US 201600089081 A1 describes a wearable sensing band that generally provides a non-intrusive way to measure a person's cardiovascular vital signs including pulse transit time and pulse wave velocity. The band includes a strap with one or more primary electrocardiography (ECG) electrodes which are in contact with a first portion of the user's body, one or more secondary ECG electrodes, and one or more pulse pressure wave arrival (PPWA) sensors. The primary and secondary ECG electrodes detect an ECG signal whenever the secondary ECG electrodes make electrical contact with the second portion of the user's body, and the PPWA sensors sense an arrival of a pulse pressure wave to the first portion of the user's body from the user's heart. The ECG signal and PPWA sensor(s) readings are used to compute at least one of a pulse transit time (PTT) or a pulse wave velocity (PWV) of the user.

The use of PPT for analyzing cardiovascular parameters has been described in the state of the art, such as in US 2015/0148663 A1 proposing a photoplethysmographic measurement apparatus, a photoplethysmographic measurement method, and an apparatus for measuring a biosignal. The photoplethysmographic measurement apparatus includes a probe, a light emitter comprising a nonelectrical light source and disposed at one end of the probe, the light emitter configured to illuminate a measurement part, and a light receiver disposed at another end of the probe and configured to detect light reflected or transmitted by the illuminated measurement part.

In WO 2014/022906 A1 a system is provided that continuously monitors cardiovascular health using an electrocardiography (ECG) source synchronized to an optical (PPG) source, without requiring invasive techniques or ongoing, large-scale external scanning procedures. The system includes an ECG signal source with electrodes contacting the skin, which generates a first set of information, and a mobile device having a camera which acts as a PPG signal source that generates a second set of information. Together with the mobile device's processor, configured to receive and process the first and second sets of information, from which the time differential of the heart beat pulmonary pressure wave can be calculated, continuous data related to cardiovascular health markers such as arterial stiffness can be determined. Variations of the ECG source may include a chest strap, a plug-in adaptor for the mobile device, or electrodes built into the mobile device.

US 2013/324859 A1 discloses a method for providing information for diagnosing arterial stiffness noninvasively using PPG. The method of the invention for assessing arterial stiffness comprises: a user information input step, characteristic point extraction step, and arterial stiffness assessment step. In particular the arterial stiffness assessment step includes the result of performing multiple linear regression analysis using the baPWV (branchial-ankle pulse wave velocity) value. PPG segmentation is conducted with the help of the PPG second derivative and the PPG pulses need to be classified to remove corrupted PPG pulses. The additional cardiovascular features, such as augmentation index and vascular age index are directly estimated from the characteristic points of the second derivative waveform. Moreover, the second derivative is used to find the position in the PPG signal of some pivotal points.

The US 2017/0238818 A1 describes a method for measuring blood pressure including illuminating by one PPG sensor included in an electronic device, the skin of a user and measuring a PPG signal based on an illumination absorption by the skin. The method also includes extracting a plurality of parameters from the PPG signal, wherein the parameters may comprise PPG features, heart rate variability (HRV) features, and non-linear features.

Elgendi (Current Cardiology Reviews, 2012, 8, 14-25) describes the use of PPG to estimate the skin blood flow using infrared light. Recent studies emphasize the potential information embedded in the PPG waveform signal and it deserves further attention for its possible applications beyond pulse oximetry and heart-rate calculation. Especially, characteristics of the PPG waveform and its derivatives may serve as a basis for evaluating vascular stiffness and aging indices.

The European patent application EP 3061392 A1 discloses a method for determining blood pressure comprising means for providing pulse wave data representative of the heart beat of a human subject, which has a body height, an age and a gender. The blood pressure of the subject is determined based on the time difference between two peaks in the same PPG pulse, the body height, age and gender.

However, all these solutions require different sensors and are not adapted to be implemented in a compact wrist-worn device. Besides, all these methods do not include individual physiological parameters of the measured subject, but only rely on the measured values.

Therefore, proceeding from the prior art, there is a need for a method to estimate the blood pressure and the arterial stiffness based on PPG signals and provide optimized algorithms for the calculation of different cardiovascular parameters based on individual physiological parameters of interest, such as height, age and other estimated parameters, such as the heart-rate. It is desired to provide a multi-functional solution that incorporates as much parameters as possible. The proposed solution should be incorporated into a compact system, such as a wrist-band or smart-watch, where additional functionalities related to the monitoring of various cardiovascular parameters could be included.

The problem is solved by providing a method for estimating one or more cardiovascular parameters in a subject, the subject having an age and a body height with the following steps:
determining the age (p_{age}) and body height (p_{height}) of the subject,
measuring at least two photoplethysmographic (PPG) signals with at least two PPG sensors at two different positions at the subject,
separating the PPG signal into PPG pulses, whereby the start point and the end point of the pulse corresponds the systolic foot of the PPG signal,
determining the heart rate of the subject (p_{HR}),
determining the systolic A_{sys} and diastolic A_{dia} peak amplitudes and their times tₛ and t_{d} and the pulse width (t_{w}),
calculating the second derivative of the PPG pulse, and determining the characteristic points a, b, c, d, and e from the second derivative of the PPG pulse, wherein
   a and e are the first and second most prominent maxima in the second derivative, respectively,
   c is the most prominent peak between the points a and e,
   b is the most prominent minimum in the second derivative and,
   d is the most prominent minimum between points c and e,
determining:
   a) the vascular age index AgIx_{PPG} using linear regression based on the characteristic points a, b, c, d, and e, age (p_{age}), body height (p_{height}) and heart rate estimation (\hat{p}_{HR}) of the subject,
   b) the pulse wave velocity PWV_{PPG-PPG} using linear regression based on the time difference between the two PPG pulses (t_{diff}), age (p_{age}), body height (p_{height}) and heart rate estimation (\hat{p}_{HR}) of the subject,
   c) blood pressure BP_{dia} and BP_{sys} using linear regression based on the peak amplitudes (tₛ and t_{d}), the pulse width (t_{w}), age (p_{age}), body height (p_{height}), heart rate estimation (\hat{p}_{HR}), the pulse wave velocity estimate (\hat{p}_{PWV}) and the vascular age index (\hat{p}_{vascAge}), and
   d) optionally the augmentation index AIx_{PPG} based on the systolic A_{sys} and diastolic A_{dia} peak amplitudes,
and outputting one or more calculated parameters.

Heart rate is estimated by the time difference between two adjacent PPG pulses.

In a preferred embodiment of the present invention the cardiovascular parameters are estimated with the following equations:

a) vascular age index AgIx_{PPG}:

AgIx_{PPG}=z₀+z₁a+z₂b+z₃c+z₄d+z₅e+z₆p_{age}+z₇p_{height}+z₈\hat{p}_{HR};

b) pulse wave velocity PWV_{PPG-PPG}:

PŴV_{PPG-PPG}=a₁t_{diff}+a₂p_{age}+a₃p_{height}+a₄p_{HR}+a₅;

c) blood pressure BPdia and BPsys:

BP_{dia}=b₀+b₁tₛ+b₂t_{d}+b₃t_{w}+b₄p_{age}+b₅p_{height}+b₆\hat{p}_{HR}+b₇\hat{p}_{PWV}+\hat{p}_{vascAge};

BP_{sys}=c₀+c₁tₛ+c₂t_{d}+c₃t_{w}+c₄p_{age}+c₅p_{height}+c₆\hat{p}_{HR}+c₇\hat{p}_{PWV}+c₈\hat{p}_{vascAge}

d) augmentation index AIx_{PPG}:

$${AIx}_{P P G} = \frac{{A}_{s y s} - {A}_{d i a}}{{A}_{s y s}} ;$$

wherein to is the pulse width, p_{age} is the age, p_{height} is the height, p_{HR} is the heart rate, \hat{p}_{HR} is the heart rate estimate, \hat{p}_{PWV} is the pulse wave velocity estimate (as estimated in step c) and \hat{p}_{vascAge} is the vascular age index estimate of the subject (as estimated in step a), t_{d} if is the time difference between the PPG pulses, A_{sys} and A_{dia} are magnitudes of the systolic and diastolic peak, respectively, a₁ to a₅, b₀ to b₈, c₀ to c₈, z₀ to z₈, represent the coefficients of the respective linear regression equation.

In a preferred configuration, the cardiovascular parameters are estimated based on at least 60 PPG pulses, preferably at least 100 PPG pulses, more preferably at least 120 PPG pulses. The estimation of 60 pulses corresponds to measurement time of approximately 1 minute (with 60 pulses per minute). Therefore, the preferred configurations refer to a measurement time of at least 1 minute (60 PPG pulses), preferably at least 1.7 minutes (100 PPG pulses), more preferably at least 2 minutes (120 PPG pulses). By combining the results obtained by every PPG pulse mediated in the measured time, this allows a more reliable estimation. In this way, if there is a corrupted PPG pulse, its effect can be smoothed if the signals are mediated over the measured time. The measurement of PPG pulses over a defined time has the advantage that the single PPG pulses do not need to be classified as it necessary in the state of the art (e.g. such as in US2013/324859A1) and this provides a more efficient algorithm.

In a further preferred configuration, the coefficients for the linear regressions are calculated based on at least 100 PPG measurements, preferably at least 150 PPG measurements, more preferably at least 200 PPG measurements. Due to the high number of independent PPG measurements, it is possible to achieve reliable coefficients for the linear regressions.

The method according to the present invention allows the estimation of blood pressure and arterial stiffness based on PPG signals. With this invention, new methods to find the characteristic points (features) that are necessary for the estimation in the PPG signal and its time derivatives are proposed. To date no algorithm to achieve this has been available. To find the characteristic points, a model for the PPG waveform is also proposed. After extraction of the features, new models which relate the extracted features to the physiological parameters of interest are provided. Unlike existing methods in the literature, the proposed models according to the present invention allow to incorporate parameters such as height, age and other estimated parameters, such as the heart-rate. In summary, based on advanced algorithms including specific anatomical data, the evaluation of several cardiovascular parameters is achieved. The evaluation of supplementary parameters, such as blood flow, blood pressure, arterial stiffness, vessel elasticity, vascular age allows a comprehensive general health assessment. This individual cardiovascular health assessment reduces the risk of misinterpretation and leads to a more precise health assessment. The measurement of new parameters using PPG sensor technology allows new health production with mobile devices, such as fitness trackers or smart watches.

It is crucial for the present invention to use two or more PPG sensors at two different positions at the subject, for determining of the cardiovascular parameters pulse wave velocity and blood pressure. The introduction of a second PPG sensor in comparison to the methods described in the prior art has the advantage that the pulse transit time (PTT, t_{diff}) can be measured (instead of being estimated), which improves the estimates for the cardiovascular parameters. The use of at least two PPG sensors allows more reliable measurements of cardiovascular parameters.

In one alternative embodiment, one PPG sensor is located at the wrist of the subject and another PPG sensor is located at the fingertip of the subject (which can be included in a mobile device, such as a mobile phone). In another alternative embodiment, one PPG sensor is located at the wrist of the subject and another PPG sensor is located at the wrist of the subject, with a defined distance to the first PPG sensor.

From the PPG pulse wave, the systolic A_{sys} and diastolic A_{dia} peak amplitudes are estimated, as well as their times tₛ and t_{d}. The determination of A_{dia} in the PPG waveform can be very difficult when the reflected wave is very small and there is no visible diastolic peak in the waveform (see FIG. 1.2). To still be able to estimate both peak positions, two different methods to model the form of the two waves were developed.

In the first method, the PPG waveform is modelled as a sum of the two pulse waves through exponential functions.

$$\begin{matrix}{y}_{pulse} ( t ) = {y}_{sys} ( t ) + {y}_{dia} ( t ) \\ = {b}_{1} {e}^{\frac{- {( t - {t}_{s} )}^{2}}{{b}_{2}}} + {b}_{3} {e}^{\frac{- {( t - {t}_{d} )}^{2}}{{b}_{4}}}\end{matrix} \qquad ( 1.1 )$$

Nonlinear regression is applied to fit the model to the PPG waveform and receive estimates of tₛ and t_{d} to find A_{sys} and A_{dia}, respectively.

The second method makes use of the fact that the maximum in the PPG waveform is the systolic peak. By modelling only the first wave with known position at the systolic peak, its exponential model is subtracted from the PPG signal and yield the remaining reflected wave,

$$\begin{matrix}{y}_{dia} ( t ) = {y}_{pulse} ( t ) + {y}_{sys} ( t ) \\ = {y}_{pulse} ( t ) - {b}_{1} {e}^{\frac{- {( t - {t}_{s} )}^{2}}{{b}_{2}}}\end{matrix} \qquad ( 1.2 )$$

whose maximal value max y_{dia}(t)=A_{dia} and and t_{d} is the corresponding diastolic time index estimate (see FIG. 1.3).

### Preprocessing of the PPG Signal

In an advantageous configuration of the present invention the raw PPG signal from the PPG sensor is processed by one or more of the following:
removal of the power line interference, preferably by a 50 Hz notch filter,
removal of high frequency noise, preferably by a moving average filter,
adjustment for the individual signal power by normalizing the signal.

In most cases, the raw PPG signal from the sensors needs to be preprocessed and interferences need to be removed. A PPG signal that is measured without any modification usually contains a visible power line interference at a frequency of 50 Hz. It is preferable to use a notch filter at 50 Hz to remove this interference.

Besides, a signal contains interference caused by movement or other disturbances. As it is important to be able to find the relevant peaks of a pulse wave (mainly systolic and diastolic peak), the signals need to be smoothed by a moving average filter. The width of the window of the moving average filter depends on the measured signal and the required precision. A good balance needs to be found between a wider window causing a smoother signal and a more narrow window which has a reduced risk of impairing the original waveform.

In the present invention it was found that a width of 10 samples is sufficient for a PPG finger signal which has very little visible disturbances and is sampled at 1000 Hz. If the signal is more disturbed, a slightly wider window is needed, which is the case for the PPG wrist signal that needs a width of 25 samples.

To adjust for the individual power of the PPG signal coming from slightly different positioning of the sensors or individual participant's conditions, the PPG signal needs to be normalized by the mean and standard deviation of the entire PPG signal. Due to better blood circulation, the PPG signal from a finger sensor has a more than ten times higher amplitude than the PPG signal from a wrist sensor.

Separation of PPG Signal into Pulses

In order to analyse each individual PPG waveform in the PPG signal and to reduce the effect of motion artefacts, the PPG signal is not examined as a whole but in sections. According to the present invention the signal is divided into individual pulses, as all features which are extracted from the PPG signal can be derived from one pulse wave. The systolic foot is the most prominent feature of a PPG pulse and can therefore be found most reliably in the PPG signal. Therefore, the PPG signal was chopped into PPG pulses at these systolic feet by finding the minima in the PPG signal. This strategy allows to analyse each pulse individually. If a few pulses are not correctly recognized, this does not have a falsifying effect on the final results for a measurement as the final parameter values are calculated by the median of all individual pulses' results.

To estimate the different cardiovascular parameters (blood pressure, pulse wave velocity, vascular age index, augmentation index and heart rate variability), the PPG waveform needs to be analysed and different features are extracted from the PPG waveform.

Blood pressure (BP):

Previous studies suggest to estimate the BP by a simple linear regression model using the extracted systolic and diastolic times of a PPG pulse:

BP_{dia}=a_{SBP}t_{dia}+b_{SBP} (1.3)

BP_{sys}=a_{DBP}t_{sys}+b_{DBP} (1.4)
Wherein a_{SBP}, b_{SBP}, a_{DBP} and b_{DBP} are coeffiients that have to be estimated based on reference values.

For the present invention this linear regression model from the literature was implemented using the two different methods to estimate the characteristic times as described above (1.1) and (1.2). Furthermore, the linear regression model was extended by incorporating the pulse width to and additional physiological and personal data as well as other estimates:

BP_{dia}=b₀+b₁tₛ+b₂t_{d}+b₃t_{w}+b₄p_{age}+b₅p_{height}+b₆\hat{p}_{HR}+b₇\hat{p}_{PWV}+\hat{p}_{vascAge} (1.5)

BP_{sys}=c₀+c₁tₛ+c₂t_{d}+c₃t_{w}+c₄p_{age}+c₅p_{height}+c₆\hat{p}_{HR}+c₇\hat{p}_{PWV}+c₈\hat{p}_{vascAge}; (1.6)
wherein t_{w} is the pulse width, p_{age} is the age, p_{height} is the height, \hat{p}_{HR} is the heart rate estimate, \hat{p}_{PWV} is the pulse wave velocity estimate and \hat{p}_{vascAge} is the vascular age index estimate of a person.

### Pulse Wave Velocity (PWV):

The PWV is estimated by the time difference between pulses of two PPG signals measured at two separately placed PPG sensors. Therefore, the time difference between the systolic feet of the signals is examined. The median time differences are used for a linear regression model to estimate the PWV:

PŴV_{PPG-PPG}=a₁t_{diff}+a₂; (1.7)

The PWV estimation algorithm is applicable for the case when two PPG signals are measured, as well as for the case of measuring one PPG and one ECG signal. Additional physiological and personal data were further included in the linear regression model:

PŴV_{PPG-PPG}=a₁t_{diff}+a₂p_{age}+a₃p_{height}+a₄p_{HR}+a₅; (1.8)
wherein t_{diff} is the time difference between the PPG pulses or between an ECG and PPG pulse, p_{age} is the age, p_{height} is the height and p_{HR} is the heart rate of a person.

It is preferred that two PPG signals are measured and the time difference between the two corresponding PPG pulses are considered. In one embodiment, one PPG sensor can be positioned at the wrist of a user and the second sensor can be positioned at the finger of a user. However, in an advantageous configuration, two PPG sensors can be positioned at the wrist of a user with a certain distance between both sensors. This allows the implementation in wrist-worn devices, such as smartwatches or fitness trackers.

Feature Extraction from Signal's Derivatives

Other features are obtained from the signal's derivatives which are calculated by the differences between adjacent samples. A moving average filter was applied to remove high frequency noise introduced by taking the derivative. To reliably find the characteristic points a to e, an algorithm to find the two most prominent maxima was developed and they were marked as a and e, respectively. The point c is then the most prominent peak between point a and e. Furthermore, point b is the most prominent minimum in the second derivative and point d is the most prominent minimum between points c and e (see FIG. 1.4).

Therefore, in a preferred embodiment of the present invention the characteristic points a, b, c, d, and e are automatically derived from the second derivative of the PPG pulse, wherein a and e are the first and second most prominent maxima in the second derivative, respectively, c is the most prominent peak between the points a and e, b is the most prominent minimum in the second derivative and, d is the most prominent minimum between points c and e.

Vascular age index (AgIx_{PPG}):

The vascular age index is a cardiovascular parameter that is calculated from the second derivative of the PPG pulse.

The state-of-the-art literature calculates a ratio of the characteristic points by

$$A g I {x}_{P P G} = 45.5 * \frac{b - c - d - e}{a} + 65.9 \qquad ( 1.9 )$$

The index describes the cardiovascular age of a person. It should be lower than the person's chronological age if their vessels aged slower than average and higher than their chronological age otherwise.

Although the modified ratio in (1.9) has been proposed, there is information on how to reliable find the characteristic points in the second derivative. For this, an algorithm was developed to find the two most prominent maxima and mark them as a and e, respectively. The point c is then the most prominent peak between point a and e. Furthermore, point b is the most prominent minimum in the second derivative and point d the most prominent minimum between point c and e.

Based the modified ratio in the new coefficients in (1.9) were found to more reliable estimate AgIx_{PPG}. Furthermore, we also developed a new linear regression model with coefficients zᵢ based on the characteristic points a, b, c, d and e was developed:

AgIx_{PPG}=z₀+z₁a+z₂b+z₃c+z₄d+z₅e; (1.10)

A more suitable linear regression model that also incorporates physiological and personal data is proposed with the current invention:

AgIx_{PPG}=z₀+z₁a+z₂b+z₃c+z₄d+z₅e+z₆p_{age}+z₇p_{height}+z₈\hat{p}_{HR}; (1.11)
wherein zᵢ are the coefficients, p_{age} is the age, p_{height} is the height, \hat{p}_{HR} is the heart rate estimate of a person.

### Augmentation Index (AIx_{PPG}):

The PPG pulse wave is not a pressure pulse wave. Thus, the augmentation index as described above be obtained directly from the PPG signal. The augmentation index is calculated with the help of the following formula:

$${AIx}_{P P G} = \frac{{A}_{s y s} - {A}_{d i a}}{{A}_{s y s}} ; \qquad ( 1.12 )$$

wherein A_{sys} and A_{dia} are magnitudes of the systolic and diastolic peak, respectively (as shown in FIG. 1.2).

The AIx_{PPG} describes the augmentation of the PPG signal from the systolic to the diastolic peak. Therefore, it is reasonable to name it PPG augmentation index.

### Heart Rate Variability (HRV):

The heart rate variability (HRV) describes the variation in the time interval between heartbeats. For simplicity, in the following, it was assumed that the pulse rate variability (PRV), estimated from the time interval between pulses measured at the PPG sensors at the wrist and fingertip, to be the same as the HRV. The PRV from the PPG signals are obtained by the locations of the systolic feet.

Therefore, in an advantageous configuration additionally the heart rate variability HRV is determined by calculating one or more of the following: the Interbeat interval in seconds, the mean heart rate in beats per minute (BPM), the standard deviation of NN intervals (SDNN) in milliseconds (ms) and the root mean square of successive differences (RMSSD), which is the square root of the mean of squares of the successive difference between adjacent time intervals. All these metrics, which are commonly used for ECG signal-based HRV analysis, are estimated from the PPG signals based on the time stamps of the detected systolic feet.

According to the present invention one or more cardiovascular parameters are calculated by measuring a PPG signal with a PPG sensor and using advanced algorithms to determine vascular age index AgIx_{PPG}, blood pressure BPdia and BPsys, pulse wave velocity PWV_{PPG-PPG} and augmentation index AIx_{PPG}.

In alternative embodiments, one or more of these cardiovascular parameters are determined with help of the advanced algorithms for the Augmentation index AIx_{PPG} (as shown in 1.1 and 1.2), Vascular age index AgIx_{PPG} (as shown in 1.10 and 1.11), Blood pressure (as shown in 1.5 and 1.6) and Pulse wave velocity PWV_{PPG-PPG} (as shown in 1.7 and 1.8).

In one configuration, only one cardiovascular parameter is measured, either the Augmentation index AIx_{PPG} is determined (as shown in 1.1 and 1.2) or only the Vascular age index AgIx_{PPG} is determined (as shown in 1.10 and 1.11), or only Blood pressure is determined (as shown in 1.5 and 1.6) or only Pulse wave velocity PWV_{PPG-PPG} is determined (as shown in 1.7 and 1.8).

In further configurations, two cardiovascular parameters are measured, either Augmentation index AIx_{PPG} (as shown in 1.1 and 1.2) and the Vascular age index AgIx_{PPG} are determined (as shown in 1.10 and 1.11). In further alternatives, additionally the Blood pressure is determined (as shown in 1.5 and 1.6) or Pulse wave velocity PWV_{PPG-PPG} (as shown in 1.7 and 1.8) or both are determined.

In further configurations Augmentation index AIx_{PPG} (as shown in 1.1 and 1.2) and Blood pressure are determined (as shown in 1.5 and 1.6). In further alternatives, additionally the Vascular age index AgIX_{PPG} is determined (as shown in 1.10 and 1.11) or Pulse wave velocity PWV_{PPG-PPG} (as shown in 1.7 and 1.8) or both are determined.

In further configurations Augmentation index AIx_{PPG} (as shown in 1.1 and 1.2) and Pulse wave velocity PWV_{PPG-PPG} (as shown in 1.7 and 1.8) are determined. In further alternatives, additionally the Vascular age index AgIx_{PPG} is determined (as shown in 1.10 and 1.11) or Blood pressure (as shown in 1.5 and 1.6) or both are determined.

In further configurations Vascular age index AgIx_{PPG} (as shown in 1.10 and 1.11) and Blood pressure are determined (as shown in 1.5 and 1.6). In further alternatives, additionally the Vascular age index AgIx_{PPG} is determined (as shown in 1.10 and 1.11) or Augmentation index AIx_{PPG} (as shown in 1.1 and 1.2) or both are determined.

In further configurations Vascular age index AgIx_{PPG} (as shown in 1.10 and 1.11) and Pulse wave velocity PWV_{PPG-PPG} are determined (as shown in 1.7 and 1.8). In further alternatives, additionally Blood pressure is determined (as shown in 1.5 and 1.6) or Augmentation index AIx_{PPG} (as shown in 1.1 and 1.2) or both are determined.

In further configurations Blood pressure (as shown in 1.5 and 1.6) and Pulse wave velocity PWV_{PPG-PPG} (as shown in 1.7 and 1.8) are determined. In further alternatives, additionally Augmentation index AIx_{PPG} (as shown in 1.1 and 1.2) is determined or Vascular age index AgIx_{PPG} (as shown in 1.10 and 1.11) or both are determined.

In a preferred configuration, the cardiovascular parameters Augmentation index AIx_{PPG} (as shown in 1.1 and 1.2), Vascular age index AgIx_{PPG} (as shown in 1.10 and 1.11), Blood pressure (as shown in 1.5 and 1.6) and Pulse wave velocity PWV_{PPG-PPG} (as shown in 1.7 and 1.8) are determined.

In a particularly preferred configuration, the cardiovascular parameters Augmentation index AIx_{PPG} (as shown in 1.1 and 1.2), Vascular age index AgIx_{PPG} (as shown in 1.11), Blood pressure (as shown in 1.5 and 1.6) and Pulse wave velocity PWV_{PPG-PPG} (as shown in 1.8) are determined.

In alternative configurations, additionally to one, two, three or four cardiovascular parameters, the heart rate variability HRV is determined by calculating one or more of the following: the median heart rate interval length in seconds, the mean heart rate in beats per minute (BPM), the standard deviation of NN intervals (SDNN) in milliseconds (ms) and the root mean square of successive differences (RMSSD), which is the square root of the mean of squares of the successive difference between adjacent time intervals.

The present invention can be applied using PGG sensors which are included in a number of different human body health monitoring devices, such as wrist-type fitness trackers, smart watches or special devices used by medical professionals. The method according to the present invention allows the detailed analysis of the cardiovascular condition of a person with the help of simple wrist-worn devices by analyzing several cardiovascular parameters.

Therefore, in an advantageous configuration of the present invention one or more calculated parameters are displayed on a human body health monitoring device, which includes at least one PPG sensor.

In an alternative configuration, one or more calculated parameters are displayed on a human body health monitoring device, which includes at least two PPG sensors, thereby allowing the evaluation of PWV by analysing the time difference between two PPG signals.

In another preferred embodiment of the present invention, an acoustic or visual signal is outputted together with the calculated parameter.

In an alternative embodiment of the present invention the calculated cardiovascular parameters are compared with prestored cardiovascular index parameters and an acoustic or visual signal is outputted, if the calculated cardiovascular parameters differ more than X % from the prestored cardiovascular index parameters, whereas X is chosen from the following values: 5, 10, 20, 30, 40, 50, 60, 70, 80, 90, 100.

### EXPERIMENTS

### Experimental Setup

The proposed methods were evaluated on 242 measurements taken in two four-weeks studies with 42 participants. The group of subjects consisted of 24 men and 18 women aged between 20 and 58, with an average age of 30.26 years. 40 of them were nonsmokers, 2 were smokers.

A measurement consisted of the two-minute recording of two PPG and one ECG signal and was followed by the recording with a clinical device to attain reference values for the cardiovascular parameters. One PPG signal was measured at the wrist, the other one at the forefinger. As a reference device, the “Mobil-O-Graph PWA” was used which is a clinical device by I.E.M. GmbH. This device works similar to a standard measurement device for blood pressure, applying a cuff to the subject's upper arm. The inflatable cuff exerts pressure onto the upper arm's brachial artery and measures not only the blood pressure, but also performs a pressure pulse wave analysis (PWA).

A measurement consisted of the two-minute recording of two PPG and one ECG signal and was followed by the recording with a clinical device to attain reference values for the cardiovascular parameters. It is assumed that those reference values are valid, as the vascular condition is not supposed to change within few minutes of rest.

### Preprocessing of the PPG Signal

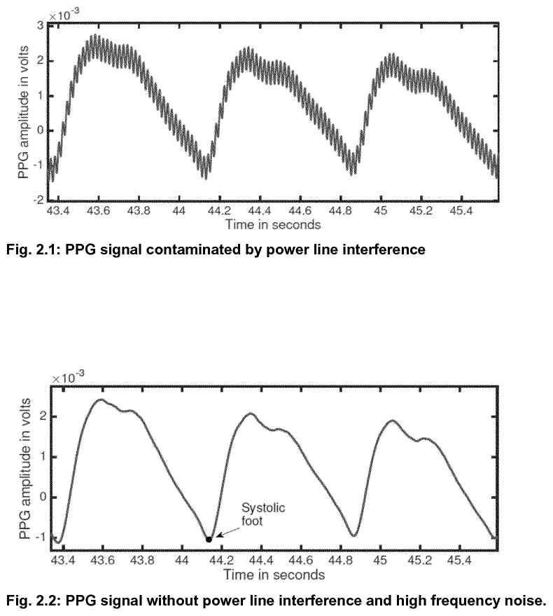

A PPG signal that is measured without any modification usually contains a visible power line interference at a frequency of 50 Hz, as displayed in FIG. 2.1. A notch filter at 50 Hz is used to remove this interference.

A PPG signal cleaned from power line interference and high frequency noise is displayed in FIG. 2.2.

At the beginning of a measurement, the participants in the study often moved their arms again to achieve a pleasant position, thus introducing very large motion artefacts. Therefore, the first ten seconds are removed, shortening the signal by 8.33%.

To adjust for the individual power of the PPG signal coming from slightly different positioning of the sensors or individual participant's conditions, the PPG signal is normalized by the mean and standard deviation of the entire PPG signal. Due to better blood circulation, the PPG signal from the finger sensor has a more than ten times higher amplitude than the PPG signal from the wrist sensor.

### Evaluation Metrics

The accuracy and reliability of the proposed algorithms was validated by comparing the estimates of those algorithms with measurements of a reference device that is clinically approved. These measurements, obtained by the Mobil-O-Graph from I.E.M. GmbH, serve as reference values and can themselves differ from the true value, as the device also has an intrinsic measurement error and thus fluctuates in its measurement values. To reduce the influence of the intrinsic measurement error of the reference device, three consecutive measurements were taken with the reference device and the median of those three values for each cardiovascular parameter were calculated.

For validation, five different metrics were calculated, the mean error, the standard deviation (STD), the mean absolute error (MAE), the mean squared error (MSE) and the root-mean-squared error (RMSE), where ŷ(i) is the estimated cardiovascular parameter of interest with length N=242 equal to the total number of measurements for all participants and y_{ref}(i) is the reference value of the cardiovascular parameter thereof:

$$mean \text{error:} MEAN = \frac{1}{N} {\sum }_{i}^{N} ( \hat{y} ( i ) - {y}_{r e f} ( i ) )$$

$$standard deviation \text{(} \text{STD} \text{):} STD = \sqrt{\frac{{{Σ}_{i}^{N} ( \hat{y} ( i ) - {y}_{r e f} ( i ) )}^{2}}{N - 1}}$$

$$mean absolute error \text{(} \text{MAE} \text{):} MAE = \frac{1}{N} {\sum }_{i}^{N} \hat{y} \vert ( i ) - {y}_{r e f} ( i ) \vert$$

$$mean squared error \text{(} \text{MSE} \text{):} \text{MSE} = \frac{1}{N} {\sum }_{i}^{N} {( \hat{y} ( i ) - {y}_{r e f} ( i ) )}^{2}$$

$$\text{root-mean-squared} error \text{(} \text{RMSE} \text{):} RMSE = \sqrt{M S E} = \sqrt{\frac{1}{N} {\sum }_{i}^{N} {( \hat{y} ( i ) - {y}_{r e f} ( i ) )}^{2}}$$

### Estimation of Linear Regression Coefficients to Estimate Cardiovascular Parameters

### 1. Augmentation Index AIx_{PPG}

The augmentation index is evaluated for the finger and wrist PPG sensors individually using both proposed methods for modelling the form of the two waves as described above (1.12). The augmentation index is given in percent and so are its estimation errors. The evaluation results are shown both for the PPG sensor at the finger and for the PPG sensor at the wrist. For the calculation according to method 1 the the PPG waveform is modeled as a sum of two pulse waves through exponential functions and nonlinear regression is applied to fit the model to the PPG waveform and receive estimates of tₛ and t_{d} to find A_{sys} and A_{d}a, respectively (1.1). For the calculation according to method 2 the first wave is modeled with known position at the systolic peak A_{sys}, and its exponential model is subtracted from the PPG signal and thereby yielding the remaining reflected wave (1.2).

TABLE 1
Evaluation of Augmentation index AIxPPG from the
finger sensor or wrist sensor using two different
methods for modeling the form of the two waves

| | MEAN | STD | MAE | MSE | RMSE |
|---|---|---|---|---|---|
| Method | (%) | (%) | (%) | (%²) | (%) |
| Finger sensor (method 1) | 8.85 | 14.71 | 13.28 | 293.92 | 17.14 |
| Finger sensor (method 2) | 12.87 | 13.07 | 14.22 | 335.88 | 18.33 |
| Wrist sensor (method 1) | 1.61 | 14.88 | 12.07 | 222.90 | 14.93 |
| Wrist sensor (method 2) | 10.82 | 13.88 | 12.86 | 308.78 | 17.57 |

The reference values from the Mobil-O-Graph were highly fluctuating even at the same measurement.

2. Vascular age index AqIx_{PPG}

The vascular age index is evaluated for the finger and wrist PPG sensors individually using the literature based ratio with fixed coefficients (1.9), the literature based ratio with updated coefficients by minimizing the error to the reference data, the new linear regression model (1.10) and the extended new linear regression model (1.11). The vascular age index is given in years (y) and so are its estimation errors. The evaluation results are shown both for the PPG sensor at the finger and for the PPG sensor at the wrist.

TABLE 2
Evaluation of Vascular age index AIxPPG with PPG finger sensor

| | MEAN | STD | MAE | MSE | RMSE |
|---|---|---|---|---|---|
| Method | (y) | (y) | (y) | (y²) | (y) |
| Literature: ratio-based | 0.49 | 12.73 | 9.86 | 161.53 | 12.71 |
| Updated ratio-based | 0.12 | 7.44 | 5.85 | 55.08 | 7.42 |
| Linear regression (LR) | 0.12 | 7.44 | 5.85 | 55.08 | 7.42 |
| Extended LR | 0.12 | 3.48 | 2.86 | 12.10 | 3.48 |

TABLE 3
Evaluation of Vascular age index AIxPPG with PPG wrist sensor

| | MEAN | STD | MAE | MSE | RMSE |
|---|---|---|---|---|---|
| Method | (y) | y) | (y) | (y²) | (y) |
| Literature: ratio-based | 1.44 | 16.99 | 13.35 | 289.35 | 17.01 |
| Updated ratio-based | — | — | — | — | — |
| Linear regression (LR) | 0.12 | 8.72 | 6.49 | 75.73 | 8.70 |
| Extended LR | 0.12 | 3.61 | 2.95 | 13.02 | 3.61 |

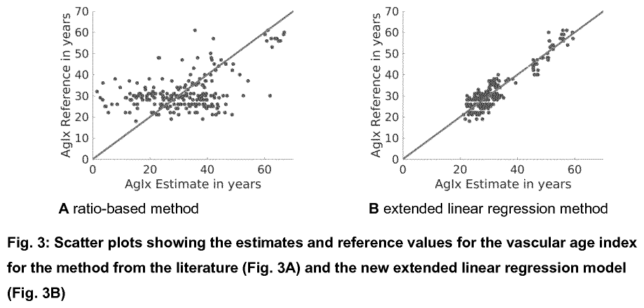

Even with the PPG signal measured at the wrist, the RMSE is only 3.61 years in relation to the vascular age value from the reference device. In FIG. 3 scatter plots are showing the estimates and reference values for the vascular age index for the method from the literature (FIG. 3A) and the new extended linear regression model (FIG. 3B).

### 3. Blood Pressure

The blood pressure is evaluated for the finger and wrist PPG sensors individually using the linear regression model from the literature (1.3) and (1.4) and using two different new implementations to find the characteristic times. Furthermore, the extended linear regression model which incorporates the participant's age and height, as well as our estimates for the heart rate, pulse wave velocity and vascular age was evaluated (1.5) and (1.6). The blood pressure is given in mmHg and so are its estimation errors.

TABLE 4
Evaluation of blood pressure with PPG finger sensor

| | MEAN | STD | MAE | MSE | RMSE |
|---|---|---|---|---|---|
| Method | (mmHg) | (mmHg) | (mmHg) | (mmHg²) | (mmHg) |
| Literature (sys) | 0 | 10.88 | 8.23 | 117.77 | 10.85 |
| Extended LR (sys) | −0.01 | 9.81 | 7.71 | 95.83 | 9.79 |
| Literature (dia) | 0 | 8.85 | 6.74 | 77.96 | 8.83 |
| Extended LR (dia) | −0.02 | 7.82 | 6.15 | 60.90 | 7.80 |

TABLE 5
Evaluation of blood pressure with PPG wrist sensor

| | MEAN | STD | MAE | MSE | RMSE |
|---|---|---|---|---|---|
| Method | (mmHg) | (mmHg) | (mmHg) | (mmHg²) | (mmHg) |
| Literature (sys) | 0 | 11.05 | 8.43 | 121.65 | 11.03 |
| Extended LR (sys) | −0.18 | 11.25 | 8.74 | 126.02 | 11.23 |
| Literature (dia) | 0 | 9.04 | 6.83 | 81.35 | 9.02 |
| Extended LR (dia) | −0.01 | 8.14 | 6.19 | 66.05 | 8.13 |

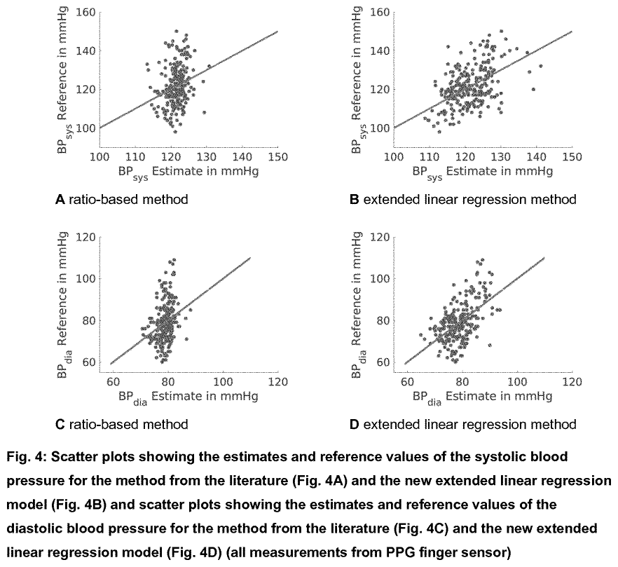

Concerning the blood pressure, the algorithms were able to achieve reasonable results of less than 10 mmHg absolute deviation on average from the reference even by using the sensor at the wrist. FIG. 4 shows scatter plots showing the estimates and reference values of the systolic blood pressure for the method from the literature (FIG. 4A) and the new extended linear regression model (FIG. 4B) and scatter plots showing the estimates and reference values of the diastolic blood pressure for the method from the literature (FIG. 4C) and the new extended linear regression model (FIG. 4D).

### 4. Pulse Wave Velocity PWV_{PPG-PPG}

The pulse wave velocity is evaluated with the time differences between ECG and PPG at the finger, ECG and PPG at the wrist, as well as between the two PPG sensors. For the estimation of pulse wave velocity two different linear regression models were applied. The first linear regression model (LR) only considers the estimated time difference as given in (1.7) and the extended linear regression model (ext. LR) additionally considers the age and height of the subject and the subject's heart rate (1.8). The pulse wave velocity is given in m/s and so are its estimation errors.

TABLE 6
Evaluation of pulse wave velocity PWV estimated from the
ECG and PPG signals at the finger, from the ECG and PPG
signals at the wrist and estimated from two PPG signals
at finger and wrist using the linear regression model
(LR) or the extended linear regression model (ext. LR)

| | MEAN | STD | MAE | MSE | RMSE |
|---|---|---|---|---|---|
| Method | (m/s) | (m/s) | (m/s) | (m/s²) | (m/s) |
| ECG-PPG finger (LR) | 0 | 0.87 | 0.62 | 0.75 | 0.87 |
| ECG-PPG finger (ext. | 0 | 0.37 | 0.29 | 0.14 | 0.37 |
| LR) | | | | | |
| ECG-PPG wrist (LR) | 0 | 0.87 | 0.62 | 0.75 | 0.87 |
| ECG-PPG wrist (ext. LR) | 0 | 0.37 | 0.29 | 0.14 | 0.37 |
| PPG-PPG (LR) | 0 | 0.83 | 0.59 | 0.69 | 0.83 |
| PPG-PPG (ext. LR) | 0 | 0.37 | 0.29 | 0.13 | 0.37 |

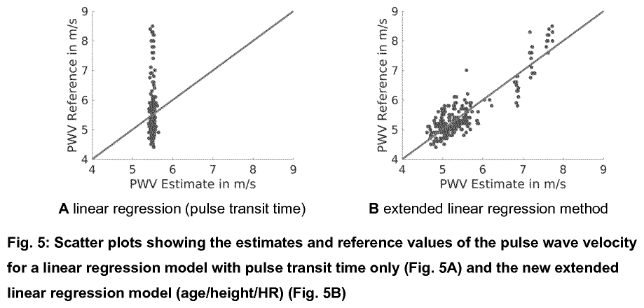

The pulse wave velocity (PWV), which is an important determinant of cardiovascular health, can be estimated best from the time difference between a PPG signal and an ECG signal and even from the time differences between the two PPG signals when using meta information of the person. FIG. 5 shows scatter plots showing the estimates and reference values of the pulse wave velocity for a linear regression model with pulse transit time only (FIG. 5A) and the new extended linear regression model (age/height/HR) (FIG. 5B).

### 5. Heart Rate Variability HRV

The heart rate variability (HRV) estimated from the PPG sensors is evaluated by estimating the reference values from the ECG signal. Four parameters of the heart rate variability were considered: the median heart rate interval length in seconds, the mean heart rate in beats per minute (BPM), the standard deviation of NN intervals (SDNN) in milliseconds (ms) and the root mean square of successive differences (RMSSD), which is the square root of the mean of the squares of the successive differences between adjacent time intervals. The evaluation results are shown both for the PPG sensor at the finger and for the PPG sensor at the wrist.

TABLE 7
Evaluation results for heart rate variability
estimated from the PPG finger sensor

| Method | MEAN | STD | MAE | MSE | RMSE |
|---|---|---|---|---|---|
| Median HR interval (ms) | −4.8 | 85.9 | 26.8 | 7.4 | 85.9 |
| Mean heart rate (BPM | −0.47 | 5.93 | 2.62 | 35.26 | 5.94 |
| SDNN (ms) | 1.9 | 58.04 | 33.85 | 3358 | 57.95 |
| Robust SDNN (ms) | 6.29 | 31.15 | 12.34 | 1005 | 31.71 |
| RMSSD (ms) | 2.52 | 67.53 | 40.20 | 4546 | 67.42 |
| Robust RMSSD (ms) | 5.29 | 32.49 | 12.55 | 1079 | 32.85 |

TABLE 8
Evaluation results for heart rate variability
estimated from the PPG wrist sensor

| Method | MEAN | STD | MAE | MSE | RMSE |
|---|---|---|---|---|---|
| Median HR interval (ms) | 172.5 | 685.8 | 181 | 498.1 | 705.8 |
| Mean heart rate (BPM | −3.26 | 13.98 | 7.70 | 205.33 | 14.33 |
| SDNN (ms) | 44.03 | 72.95 | 63.39 | 7226 | 85.01 |
| Robust SDNN (ms) | 15.06 | 29.02 | 19.11 | 1065 | 32.63 |
| RMSSD (ms) | 43.73 | 82.51 | 71.02 | 8666 | 93.09 |
| Robust RMSSD (ms) | 19.72 | 39.61 | 25.82 | 1950 | 44.16 |

The results for the heart rate variability have to be carefully considered, because the reference HRV had to be estimated from the ECG signal, as the reference device did not provide reference values for the HRV. Thus, not only small errors in the estimated HRV from the PPG signals result in strong deviations from the reference, but also errors while estimating the reference HRV from the ECG would result in erroneous evaluation results. Therefore, the SDNN and RMSSD from the ECG and PPG signals were also robustly estimated which achieved better results.

The analysis of the cardiovascular parameter estimation has shown that there are multiple cardiovascular parameters that can be estimated with reasonable deviation from the reference. To conclude, the simple and low-cost PPG signal contains useful information about a person's cardiovascular health that lay far beyond the pulse rate, which is currently the most common extracted feature. The novel algorithms according to the present invention are capable of estimating cardiovascular parameters with only a slight deviation from the reference values even in case of a PPG sensor at the wrist.

1. A method for determining one or more cardiovascular parameters in a subject, the subject having an age (p_{age}) and a body height (p_{height}), the method comprising:
determining the age (p_{age}) and the body height (p_{height}) of the subject;
measuring at least two photoplethysmographic (PPG) signals with at least two PPG sensors at two different positions at the subject;
separating the at least two PPG signals into PPG pulses, whereby a start point and an end point of each of the PPG pulses are successive systolic feet of the respective PPG signal;
determining a heart rate (p_{HR}) of the subject;
determining systolic and diastolic peak amplitudes (A_{sys} and A_{dia}) and times (t_{g} and t_{d}) and a pulse width (t_{w});
calculating a second derivative of each of the PPG pulses:
determining characteristic points a, b, c, d and e of the second derivative of each of the PPG pulses, wherein
a and e are the first and second most prominent maxima in the second derivative, respectively,
c is the most prominent peak between a and e,
b is the most prominent minimum in the second derivative and,
d is the most prominent minimum between c and e; and
determining one or more cardiovascular parameters as follows:
a) a vascular age index AgIx_{PPG} using linear regression based on the characteristic points a, b, c, d and e, the age (p_{age}), the body height (p_{height}) and a heart rate estimation (\hat{p}_{HR}) of the subject,
b) a pulse wave velocity PWV_{PPG-PPG} using linear regression based on a time difference between two of the PPG pulses (t_{diff}), the age (p_{age}), the body height (p_{height}) and the heart rate estimation (\hat{p}_{HR}) of the subject,
c) diastolic and systolic blood pressure BP_{dia} and BP_{sys} using linear regression based on the systolic and diastolic peak times (tₛ and t_{d}), the pulse width (t_{w}), the age (p_{age}), the body height (p_{height}), the heart rate estimation (\hat{p}_{HR}), a pulse wave velocity estimate (\hat{p}_{PWV}) and a vascular age index estimate (\hat{p}_{vascAge}); and
d) optionally an augmentation index AIx_{PPG} based on the systolic and diastolic peak amplitudes (A_{sys} and A_{dia}); and
outputting the one or more cardiovascular parameters.

2. The method of claim 1, wherein the one or more cardiovascular parameters are determined with the following equations:
a) the vascular age index AgIx_{PPG}:
AgIx_{PPG}=z₀+z₁a+z₂b+z₃c+z₄d+z₅e+z₆p_{age}+z₇p_{height}+z₈\hat{p}_{HR};
b) the pulse wave velocity PWV_{PPG-PPG}:
PWV_{PPG-PPG}=a₁t_{diff}+a₂p_{age}+a₃p_{height}+a₄p_{HR}+a₅;
c) the diastolic and systolic blood pressure BPdia and BPsys:
BP_{dia}=b₀+b₁tₛ+b₂t_{d}+b₃t_{w}+b₄p_{age}+b₅p_{height}+b₆\hat{p}_{HR}+b₇\hat{p}_{PWV}+b₈
BP_{sys}=c₀+c₁tₛ+c₂t_{d}+c₃t_{w}+c₄p_{age}+c₅p_{height}+c₆\hat{p}_{HR}+c₇\hat{p}_{PWV}+c₈\hat{p}_{vascAge};
and
d) the augmentation index AIx_{PPG}:
$${AIx}_{P P G} = \frac{{A}_{s y s} - {A}_{d i a}}{{A}_{s y s}} ;$$
wherein a₁ to a₅, b₀ to b₈, c₀ to c₈ and z₀ to z₈ represent coefficients of the respective linear regression equation.

3. The method of claim 1, wherein the one or more cardiovascular parameters are determined based on at least 60 PPG pulses.

4. The method of claim 1, wherein coefficients of the linear regressions are calculated based on at least 100 PPG measurements.

5. The method of claim 1, wherein a heart rate variability HRV is determined by calculating one or more of the following:
an interbeat interval in seconds, a mean heart rate in beats per minute (BPM), a standard deviation of NN intervals (SDNN) in milliseconds (ms) and a root mean square of successive differences (RMSSD), which is a square root of a mean of squares of successive differences between adjacent time intervals.

6. The method of claim 1, wherein the characteristic points a, b, c, d and e are automatically derived from the second derivative of each of the PPG pulses.

7. The method of claim 1, wherein the systolic and diastolic peak amplitudes (A_{sys} and A_{dia}) and times (tₛ and t_{d}) are determined by one of the following methods:
modeling a PPG waveform as a sum of two pulse waves through exponential functions and applying nonlinear regression to fit the model to the PPG waveform and receive estimates of tₛ and t_{d} to find A_{sys} and A_{dia}, respectively, or
modeling a first wave with known position at A_{sys}, and subtracting the model from the PPG signal to yield a remaining reflected wave.

8. The method of claim 1, wherein a raw PPG signal from the at least two PPG sensors is processed by one or more of the following:
removal of a power line interference,
removal of high frequency noise, and
adjustment for an individual signal power by normalizing the raw PPG signal.

9. The method of claim 1, wherein the one or more cardiovascular parameters are displayed on a human body health monitoring device, comprising a PPG sensor.

10. The method of claim 1, wherein an acoustic or visual signal is outputted together with the one or more cardiovascular parameters.

11. The method of claim 1, wherein the one or more cardiovascular parameters are compared with prestored cardiovascular index parameters and, if the one or more cardiovascular parameters differ by more than X % from the prestored cardiovascular index parameters, then an acoustic or visual signal is outputted, wherein X is chosen from the following values: 5, 10, 20, 30, 40, 50, 60, 70, 80, 90 and 100.
